(19) United States
(12) Patent Application Publication
(10) Pub. No.: US 2025/0101427 A1
(43) Pub. Date: Mar. 27, 2025

(54) COMPOSITIONS AND METHODS FOR USING PURIFIED HUMAN RNA EDITING ENZYMES

(71) Applicant: The Regents of the University of California, Oakland, CA (US)

(72) Inventors: Catriona Jamieson, San Diego, CA (US); Gabriel Pineda, San Diego, CA (US); Eduardo Reynoso Moreno, San Diego, CA (US); Jessica Pham, San Diego, CA (US); Luisa Ladel, San Diego, CA (US)

(21) Appl. No.: 18/290,961

(22) PCT Filed: Jul. 22, 2022
(86) PCT No.: PCT/US2022/038010

§ 371 Date: Jan. 22, 2024

Related U.S. Application Data
(60) Provisional application No. 63/224,818, filed on Jul. 22, 2021.

(51) Int. Cl. C12N 15/113

(52) U.S. Cl. CPC C12N 15/1131; C12N 2310/11; C12N 2310/531

(57) ABSTRACT

In alternative embodiments, provided are methods for eradicating or reducing the in vivo numbers of cancer stem cells comprising administering to an individual in need thereof an ADAR1 (adenosine deaminase associated with RNA1) inhibiting agent, wherein the ADAR1 inhibiting agent reduces, or significantly reduces, ADAR1 Nano-luc reporter activity in cell lines and in human cancer stem cell assays. In alternative embodiments, provided are methods for inhibiting an RNA virus or a retrovirus, optionally a SARs-COV-2 virus, comprising lentiviral ADAR1 overexpression and in vivo administration, optionally intravenous (IV) administration, of a lentiviral ADAR1 transduced stem cell, optionally the stem cell is a cord blood CD34+ cell or a mesenchymal stromal cell.

### RELATED APPLICATIONS

This Patent Convention Treaty (PCT) International Application claims the benefit of priority under 35 U.S.C. § 119 (e) of U.S. Provisional Serial Application No. 63/224,818, Jul. 22, 2021. The aforementioned application is expressly incorporated herein by reference in its entirety and for all purposes. All publications, patents, patent applications cited herein are hereby expressly incorporated by reference for all purposes.

### TECHNICAL FIELD

This invention generally relates to molecular biology and medicine. In alternative embodiments, provided are methods for eradicating or reducing the in vivo numbers of cancer stem cells comprising administering to an individual in need thereof an ADAR1 (adenosine deaminase associated with RNA1) inhibiting agent, wherein the ADAR1 inhibiting agent reduces, or significantly reduces, ADAR1 Nano-luc reporter activity in cell lines and in human cancer stem cell assays. In alternative embodiments, provided are methods for inhibiting an RNA virus or a retrovirus, optionally a SARs-COV-2 virus, comprising lentiviral ADAR1 overexpression and in vivo administration, optionally intravenous (IV) administration, of a lentiviral ADAR1 transduced stem cell, optionally the stem cell is a cord blood CD34+ cell or a mesenchymal stromal cell.

### BACKGROUND

Anti-viral deamination by ADAR1 induces adenosine to inosine (A-to-I) editing that restricts replication of RNA viruses, such as coronaviruses and influenza as well as retroviruses like HIV. Targeted base editing by ADAR1 has also emerged as a potent means to introduce single nucleotide changes in RNA to alter splice acceptor sites and transcript susceptibility to microRNA targeting and ultimately changes in translation. Moreover, Z alpha DNA binding by ADAR1 may alter the epigenome within select Alu-containing regions while Z alpha RNA binding may induce changes in transcript stability. Hyper-editing by ADAR1 induces RNA alterations in survival and stem cell transcripts, lncRNA and primary microRNA, primarily in the context of double stranded RNA loops formed by Alu sequences, and promotes therapeutic resistance in cancer stem cells as well as self-renewal of normal human hematopoietic stem cells.

As an innate immune anti-viral deaminase, ADAR1 is transcriptionally activated following inflammatory cytokine signaling through JAK2/STAT and interferon α, β and γ signaling. Thus, selective JAK2 as well as STAT3 inhibition prevents ADAR1 activation.

### SUMMARY

In alternative embodiments, provided are methods for inhibiting an RNA virus or a retrovirus, optionally a SARs-COV-2 virus, optionally inhibiting an RNA virus or a retrovirus in an individual in need thereof in vivo, comprising in vivo lentiviral ADAR1 expression or overexpression and in vivo administration, optionally intravenous (IV) administration, of a lentiviral ADAR1 transduced stem cell, optionally the stem cell is a cord blood CD34+ cell or a mesenchymal stromal cell.

In alternative embodiments, provided are methods for inhibiting replication of an RNA virus or a retrovirus, optionally a SARs-COV-2 virus, optionally inhibiting an RNA virus or a retrovirus in an individual in need thereof in vivo, comprising in vivo delivering or administration of an ADAR1 catalytic domain nanoprotein, optionally delivering or administering the ADAR1 catalytic domain nanoprotein contained in or formulated in a liposome, lipid nanoparticle (LNP), or nanoliposome, optionally delivering the ADAR1 catalytic domain nanoprotein by intravenous administration or by inhalation.

In alternative embodiments, provided are methods for inhibiting replication of an RNA virus or a retrovirus, optionally a SARs-COV-2 virus, optionally inhibiting an RNA virus or a retrovirus in an individual in need thereof in vivo, comprising in vivo delivering or administration of an ADAR1 full length nanoprotein, wherein optionally the ADAR1 full length nanoprotein is contained in or formulated with a liposome, lipid nanoparticle (LNP), or nanoliposome, and optionally the ADAR1 full length nanoprotein is delivered or administered by intravenous administration or by inhalation.

In alternative embodiments, provided are methods for inhibiting replication of an RNA virus or a retrovirus, optionally a SARs-COV-2 virus, optionally inhibiting an RNA virus or a retrovirus in an individual in need thereof in vivo, comprising in vivo delivering or administration of an ADAR1 Z alpha domain-deleted nanoprotein delivery, wherein optionally the ADAR1 Z alpha domain-deleted nanoprotein is contained in or formulated with a liposome, lipid nanoparticle (LNP), or nanoliposome, and optionally the ADAR1 Z alpha domain-deleted nanoprotein is delivered or administered by intravenous administration or by inhalation.

In alternative embodiments, provided are uses of a lentiviral ADAR1 transduced stem cell, optionally cord blood CD34+ cell or a mesenchymal stromal cell, for inhibiting an RNA virus or a retrovirus, optionally a SARs-COV-2 virus, optionally inhibiting an RNA virus or a retrovirus in an individual in need thereof in vivo, comprising in vivo lentiviral ADAR1 expression or overexpression and in vivo administration, optionally intravenous (IV) administration, of.

In alternative embodiments, provided are uses of an ADAR1 catalytic domain nanoprotein contained in or formulated in a liposome, lipid nanoparticle (LNP), or nanoliposome, and optionally the ADAR1 catalytic domain nanoprotein is delivered by intravenous administration or by inhalation, for inhibiting replication of an RNA virus or a retrovirus, optionally a SARs-COV-2 virus, optionally inhibiting an RNA virus or a retrovirus in an individual in need thereof in vivo, comprising in vivo delivering or administration of an ADAR1 catalytic domain nanoprotein.

In alternative embodiments, provided are uses of an ADAR1 full length nanoprotein is contained in or formulated with a liposome, lipid nanoparticle (LNP), or nanoliposome for inhibiting replication of an RNA virus or a retrovirus, optionally a SARs-COV-2 virus, optionally inhibiting an RNA virus or a retrovirus in an individual in need thereof in vivo, comprising in vivo delivering or administration of an ADAR1 full length nanoprotein, and optionally the ADAR1 full length nanoprotein is delivered or administered by intravenous administration or by inhalation.

In alternative embodiments, provided are uses of an ADAR1 Z alpha domain-deleted nanoprotein is contained in or formulated with a liposome, lipid nanoparticle (LNP), or nanoliposome for inhibiting replication of an RNA virus or a retrovirus, optionally a SARs-COV-2 virus, optionally inhibiting an RNA virus or a retrovirus in an individual in need thereof in vivo, comprising in vivo delivering or administration of an ADAR1 Z alpha domain-deleted nanoprotein delivery, and optionally the ADAR1 Z alpha domain-deleted nanoprotein is delivered or administered by intravenous administration or by inhalation.

In alternative embodiments, provided are methods for eradicating or reducing the in vivo numbers of cancer stem cells comprising administering to an individual in need thereof an ADAR1 (adenosine deaminase associated with RNA1) inhibiting agent, wherein the ADAR1 inhibiting agent reduces, or significantly reduces, ADAR1 Nano-luc reporter activity in cell lines and in human cancer stem cell assays.

In alternative embodiments of methods as provided herein:
the ADAR1 inhibiting agent comprises a JAK2 inhibitor, and optionally the JAK2 inhibitor comprises fedratinib, or INREBIC™, or ruxolitinib, or JAKAFI™;
the ADAR1 inhibiting agent comprises a STAT3 inhibitor;
the ADAR1 inhibiting agent comprises 8-aza-adenosine, a nucleoside analog or an integrase inhibitor;
the ADAR1 inhibiting agent comprises raltegravir (or ISENTRESS™) or dolutegravir (or TIVICAY™);
the ADAR1 inhibiting agent comprises a lentiviral shRNA ADAR1 knockdown vector;
the ADAR1 inhibiting agent comprises a lentiviral ADAR1 mutant vector;
the ADAR1 inhibiting agent comprises a lentiviral ADAR1 Z alpha domain deleted vector;
the ADAR1 inhibiting agent comprises an interferon inhibitory compound;
the ADAR1 inhibiting agent comprises lentiviral ADAR1 or lentiviral ADAR1 shRNA;
the ADAR1 inhibiting agent comprises a recombinant human full length ADAR1 protein;
the ADAR1 inhibiting agent comprises a recombinant human ADAR1 catalytic domain protein;
the ADAR1 inhibiting agent comprises a recombinant human Z alpha domain deleted ADAR1 protein; and/or
the ADAR1 inhibiting agent comprises a JAK2-expressing vector, optionally a retroviral or a lentiviral JAK2 expression vector, optionally a retroviral or a lentiviral JAK2 overexpression vector.

In alternative embodiments, provided are methods for identifying an ADAR1 agonist comprising contacting an ADAR1 Nano-luc reporter interferon-responsive cell line and an interferon cell line a candidate ADAR1 agonist; and, optionally the candidate ADAR1 agonist comprises a recombinant human full length ADAR1, and optionally the candidate ADAR1 agonist comprises a recombinant human ADAR1 catalytic domain; and optionally the candidate ADAR1 agonist comprises a recombinant human Z alpha domain deleted ADAR1; and optionally the candidate ADAR1 agonist comprises a lentiviral JAK2 overexpression vector.

In alternative embodiments, provided are stably transduced human non-interferon responsive cell lines having contained therein a lentiviral ADAR1 overexpression vector and a Nano-luc reporter for the purpose of detecting RNA virus inhibition, wherein optionally the RNA virus is SARS-COV-2, or influenza A or B.

In alternative embodiments, provided are stably transduced human interferon responsive cell lines having contained therein a lentiviral ADAR1 overexpression vector and a Nano-luc reporter for the purpose of detecting RNA virus inhibition following infection with an RNA virus or retrovirus, wherein optionally the virus is SARS-COV-2, or influenza A or B, or HIV.

In alternative embodiments, provided are uses of an ADAR1 inhibiting agent for eradicating or reducing the in vivo numbers of cancer stem cells, wherein the ADAR1 inhibiting agent is administered to an individual in need thereof, and optionally the ADAR1 inhibiting agent comprises: a JAK2 inhibitor, and optionally the JAK2 inhibitor comprises fedratinib, or INREBIC™, or ruxolitinib, or JAKAFI™; a STAT3 inhibitor; a 8-aza-adenosine, a nucleoside analog or an integrase inhibitor; raltegravir (or ISENTRESS™) or dolutegravir (or TIVICAY™); a retroviral or a lentiviral shRNA ADAR1 knockdown vector; a retroviral or a lentiviral ADAR1 mutant-expressing vector; a lentiviral ADAR1 Z alpha domain deleted vector; an interferon inhibitory compound; a lentiviral ADAR1 or lentiviral ADAR1 shRNA; a recombinant human full length ADAR1 protein; a recombinant human ADAR1 catalytic domain protein; a recombinant human Z alpha domain deleted ADAR1 protein; and/or a JAK2-expressing vector, optionally a retroviral or a lentiviral JAK2 expression vector, optionally a retroviral or a lentiviral JAK2 overexpression vector.

In alternative embodiments, provided are ADAR1 inhibiting agents for use in eradicating or reducing the in vivo numbers of cancer stem cells, wherein the ADAR1 inhibiting agent is administered to an individual in need thereof, and optionally the ADAR1 inhibiting agent comprises: a JAK2 inhibitor, and optionally the JAK2 inhibitor comprises fedratinib, or INREBIC™, or ruxolitinib, or JAKAFI™; a STAT3 inhibitor; a 8-aza-adenosine, a nucleoside analog or an integrase inhibitor; raltegravir (or ISENTRESS™) or dolutegravir (or TIVICAY™); a retroviral or a lentiviral shRNA ADAR1 knockdown vector; a retroviral or a lentiviral ADAR1 mutant-expressing vector; a lentiviral ADAR1 Z alpha domain deleted vector; an interferon inhibitory compound; a lentiviral ADAR1 or lentiviral ADAR1 shRNA; a recombinant human full length ADAR1 protein; a recombinant human ADAR1 catalytic domain protein; a recombinant human Z alpha domain deleted ADAR1 protein; and/or a JAK2-expressing vector, optionally a retroviral or a lentiviral JAK2 expression vector, optionally a retroviral or a lentiviral JAK2 overexpression vector.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-K illustrate an exemplary process for the expression and purification of recombinant human ADAR1 Catalytic Domain (hADAR1 CD) in a BJ2168 yeast expression system:

FIG. 1A graphically illustrates hADAR1 Catalytic Domain (CD) codon optimization for expression in yeast;

FIG. 1B illustrates the hADAR1 Catalytic Domain (CD) amino acid sequence (SEQ ID NO:1), where the colored (or lighter colored) amino acids have been deleted in the Δloop construct;

FIG. 1C illustrates a schematic of the exemplary pEG(KT) GST-TEV-hADAR1 Catalytic Domain (CD) and pEG(KT) GST-TEV-hADAR1 CD Δloop vector maps;

FIG. 1D illustrates a schematic representation of an exemplary Galactose-inducible expression system;

FIG. 1E illustrates an image of a Coomassie Blue stained α-ADAR1 Western Blot confirming Galactose-inducible expression of GST-tagged hADAR1 Catalytic Domain (CD);

FIG. 1F illustrates a schematic of an exemplary workflow showing steps involved in protein purification from yeast cell extract;

FIG. 1G illustrates an image of a Coomassie Blue stained gel showing successful cleavage of GST tag by TEV enzyme;

FIG. 1H illustrates an image of a silver stained gel demonstrating purity of the hADAR1 Catalytic Domain (CD) protein product after final purification step;

FIG. 1I graphically illustrates data from a Size Exclusion Chromatography of purified hADAR1 Catalytic Domain (CD) using a SUPERDEX 200 10/300 GL™ gel filtration column;

FIG. 1J graphically illustrates data from a protein mass determination of purified hADAR1 Catalytic Domain (CD) protein product via mass spectrometry; and

FIG. 1K graphically illustrates data from an analytical Ultracentrifugation of purified hADAR1 Catalytic Domain (CD) demonstrating purity of the final protein product, as discussed in further detail in Example 1, below

FIG. 2A-C illustrate an exemplary process for the expression and purification of recombinant human full-length ADAR1 Catalytic Domain (hADAR1 CD) in a BJ2168 yeast expression system:

FIG. 2A illustrates a schematic of an exemplary p424 10×His-tagged full-length ADAR1 vector map;

FIG. 2B illustrates a schematic representation of an exemplary Galactose-inducible expression system; and

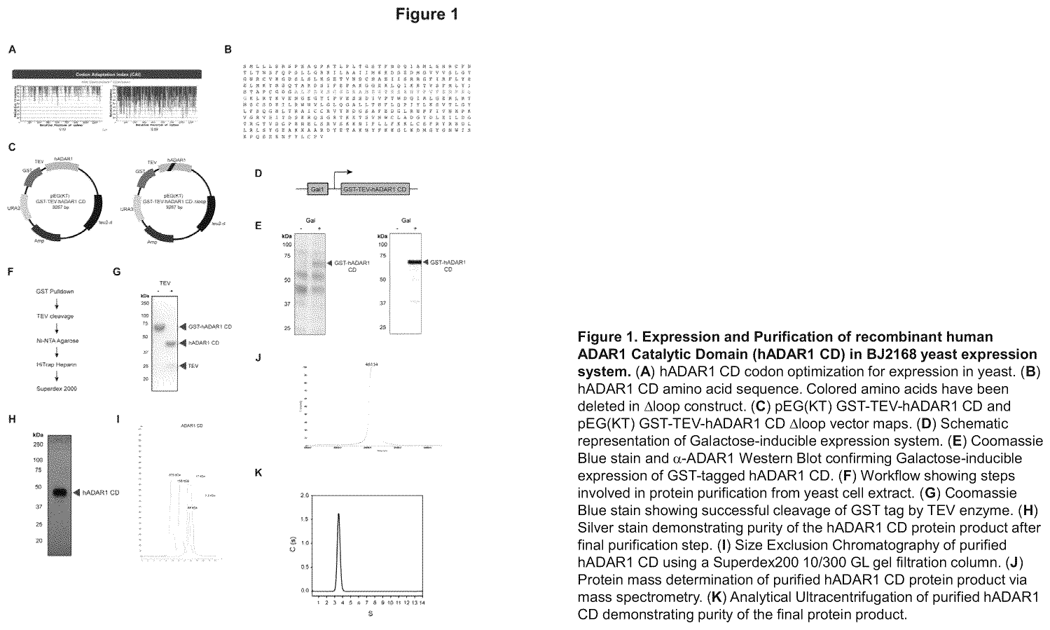

FIG. 1C graphically illustrates data from a Coomassie Blue stained gel confirming Galactose-inducible expression of 10×His-tagged full-length ADAR1, as discussed in further detail in Example 1, below

FIG. 3A-C illustrate an exemplary nano-luciferase-based RNA editase activity reporter assay in vitro:

FIG. 3A illustrates a schematic representation of an exemplary nano-luciferase reporter design;

FIG. 3B illustrates a schematic representation of an exemplary lentiviral NanoLuciferase RNA editase reporter expression vector;

FIG. 3C upper panel graphically illustrates a nanoLuciferase activity assay showing concentration-dependency and specificity for ADAR1 editase activity in HEK293T cells after co-transfection with FLAG-tagged ADAR1 constructs and NanoLuciferase reporter;

FIG. 3C lower panel illustrates an image of an a-FLAG Western Blot analysis demonstrating increasing FLAG-ADAR protein levels;

FIG. 3D upper panel illustrates a schematic representation of an exemplary NanoLuciferase activity assay comparing ADAR1 RNA editase activity in K562 cells after co-transduction with pCDH/ADAR1 and NanoLuciferase reporter; and

FIG. 3D lower panel illustrates an image of an α-ADAR1 Western Blot analysis demonstrating equal ADAR1 protein levels for all conditions (left) and RT-PCR showing equal expression of NanoLuciferase reporter for all conditions as well as in parental un-transduced K562 cells as a control (right), as discussed in further detail in Example 1, below

FIG. 4A-B illustrate an exemplary nano-luciferase-based RNA editase activity reporter assay in vitro:

FIG. 4A upper panel graphically illustrates a NanoLuciferase activity assay comparing ADAR1 RNA editase activity in K562 cells after co-transduction with pCDH vector/ADAR1 and NanoLuciferase reporter;

FIG. 4A lower panel illustrates an image of an α-ADAR1 Western Blot analysis demonstrating equal ADAR1 protein levels for all conditions (left) and RT-PCR showing equal expression of NanoLuciferase reporter for all conditions as well as in parental un-transduced K562 cells as a control (right); and

FIG. 4B illustrates an image of an IVIS™ imaging of 6.5-week-old mice after neonatal intrahepatic transplantation with K562 cells co-transduced with pCDH/wildtype ADAR1/editase-deficient ADAR1 E912A and Nano-luciferase reporter demonstrating in vivo visualization of RNA editase activity, as discussed in further detail in Example 1, below

FIG. 5A-D illustrate an exemplary assay for stable lentiviral overexpression of ADAR1 wildtype (WT) and ADAR1 mutants after shADAR1 knockdown:

FIG. 5A graphically illustrates the total ADAR1 (left) and ADAR1 p150 isoform (right) expression levels in TF1a cells after transduction with shSchramble and shADAR1 as shown by qPCR (normalized to HPRT);

FIG. 5B illustrates an image of a Western Blot analysis showing protein levels of ADAR1 in TF1a cells after transduction with shSchramble and shADAR1;

FIG. 5C illustrates schematic representations of an exemplary lentiviral expression vectors of HA-tagged, shADAR1-resistant (shR) ADAR1 wildtype, ADAR1 editase-deficient mutant E921A, ADAR1 DNA-binding domain-deficient mutant dZa and ADAR1 mutant E912A dZa constructs; and

FIG. 5D (left) graphically illustrates NanoLuciferase activity assay comparing ADAR1 RNA editase activity in TF1a cells after co-transduction with pCDH/ADAR1 shR vectors and NanoLuciferase reporter into the background of shRNA-mediated ADAR1 knockdown; and

FIG. 5D (right) graphically illustrates an a-HA Western Blot analysis demonstrating similar ADAR1 protein levels for all conditions; as discussed in further detail in Example 1, below

FIG. 6A-D illustrate data showing the involvement of ADAR1 in the JAK/STAT pathway, and demonstrating that JAK inhibitors such as ruxolitinib and fedratinib can be used as ADAR-1 inhibiting agents:

FIG. 6A graphically illustrates ADAR1 p150 isoform expression level in TF1a cells as shown by qPCR 16 hrs after treatment with PBS (control) or Interferon alpha (normalized to HPRT);

FIG. 6B illustrates an image of a Western Blot analysis of TF1a cells depicting protein levels of ADAR1 and various members of the JAK/STAT pathway 16 hrs after treatment with PBS (control) or Interferon alpha;

FIG. 6C illustrates an image of a Western Blot analysis of secondary AML (patient 672) CD34+ cells showing protein levels of ADAR1, STAT3 and phospho-STAT3 Y705 16 hrs after treatment with PBS (control), interferon alpha, beta or gamma; and

FIG. 6D illustrates an image of a Western blot analysis of secondary AML (patient 255) CD34+ cells treated with FDA approved JAK2 inhibitors (ruxolitinib and fedratinib) compared with a JAK3 inhibitor (FM-381) at concentrations of 1 nM, 10 nM, and 100 nM, as discussed in further detail in Example 1, below

Like reference symbols in the various drawings indicate like elements.

### DETAILED DESCRIPTION

In alternative embodiments, provided are methods for the purification and production of human functional anti-viral RNA editing enzymes, ADAR1 (adenosine deaminase associated with RNA1), and related lentiviral vectors, editing reporters and compounds as well as methods of use relating to the discovery of anti-viral compounds, stem cell expansion, inhibition of cancer stem cells and selective RNA base editing as well as inhibition of RNA viruses including SARS COV-2 and retroviruses. In alternative embodiments, provided are methods for producing large amounts of recombinant human full length ADAR1, Z alpha binding domain deleted ADAR1, and the catalytic domain of ADAR1.

In alternative embodiments, provided are methods for purifying human full length ADAR1, Z alpha domain deleted ADAR1 and the catalytic domain of human ADAR1.

In alternative embodiments, provided are methods for producing lentiviral ADAR1 Nano-luc reporter transduced interferon responsive and unresponsive cell lines with ADAR1 overexpression and shRNA knockdown for the purposes of screening for anti-viral compounds capable of inhibiting replication of RNA viruses and retroviruses.

In alternative embodiments, provided are methods for identifying ADAR1 antagonists, including lentiviral ADAR1 shRNA knockdown, mutant and Z alpha domain deleted ADAR1 vectors, capable of inhibiting cancer stem cells.

In alternative embodiments, provided are methods for detecting ADAR1 agonists, including lentiviral ADAR1 overexpression vectors capable of enhancing stem cell survival and self-renewal, and vectors having anti-viral activity.

### ADAR1 In Vivo Delivery and Gene Delivery Vehicles

In alternative embodiments, methods as provided herein comprise inhibiting an RNA virus or a retrovirus, optionally a SARs-COV-2 virus, comprising a viral, for example, a lentiviral or adeno-associated virus (AAV) mediated, ADAR1 overexpression and in vivo administration, optionally by intravenous (IV) administration, of a viral, for example, a lentiviral- or AAV-ADAR1 transduced stem cell, wherein optionally the stem cell is a cord blood CD34+ cell or a mesenchymal stromal cell. In alternative embodiments, the viral vectors are delivered to a cell or cells in vitro, ex vivo, or in vivo, for example, as ADAR1 delivery vehicles.

In alternative embodiments, expression vehicle, vector, recombinant virus, or equivalents used to practice methods as provided herein are or comprise: an adeno-associated virus (AAV), a lentiviral vector or an adenovirus vector; an AAV serotype AAV5, AAV6, AAV8 or AAV9; a rhesus-derived AAV, or the rhesus-derived AAV AAVrh.10hCLN2; an organ-tropic AAV; and/or an AAV capsid mutant or AAV hybrid serotype. In alternative embodiments, the AAV is engineered to increase efficiency in targeting a specific cell type that is non-permissive to a wild type (wt) AAV and/or to improve efficacy in infecting only a cell type of interest. In alternative embodiments, the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid. It is well known in the art how to engineer an adeno-associated virus (AAV) capsid in order to increase efficiency in targeting specific cell types that are non-permissive to wild type (wt) viruses and to improve efficacy in infecting only the cell type of interest; see for example, Wu et al., Mol. Ther. 2006 September; 14 (3): 316-27. Epub 2006 Jul. 7; Choi, et al., Curr. Gene Ther. 2005 June; 5 (3): 299-310.

For example, the rhesus-derived AAV AAVrh. 10hCLN2 or equivalents thereof can be used, wherein the rhesus-derived AAV may not be inhibited by any pre-existing immunity in a human; see for example, Sondhi, et al., Hum Gene Ther. Methods. 2012 October; 23 (5): 324-35, Epub 2012 Nov. 6; Sondhi, et al., Hum Gene Ther. Methods. 2012 Oct. 17; teaching that direct administration of AAVrh. 10hCLN2 to the CNS of rats and non-human primates at doses scalable to humans has an acceptable safety profile and mediates significant payload expression in the CNS.

Because adeno-associated viruses (AAVs) are common infective agents of primates, and as such, healthy primates carry a large pool of AAV-specific neutralizing antibodies (NAbs) which inhibit AAV-mediated gene transfer therapeutic strategies, the methods as provided herein also comprise screening of patient candidates for AAV-specific NAbs prior to treatment, especially with the frequently used AAV8 capsid component, to facilitate individualized treatment design and enhance therapeutic efficacy; see, for example, Sun, et al., J. Immunol. Methods. 2013 Jan. 31; 387 (1-2): 114-20, Epub 2012 Oct. 11.

Any lentiviral vectors can be used to practice methods as provided herein, for example, to in vitro, ex vivo, or in vivo deliver ADAR1 or cells such as stem cells expressing ADAR1, for example, as described in USPNs 11,299,752; 11,208,669; 11,078,495; 11,007,209; and 10,954,530.

### Pharmaceutical Compositions and Formulations

In alternative embodiments, ADAR1 inhibiting agents, including drugs, vectors, liposomes, lipid nanoparticles (LNP), nanoliposomes, or nanoparticles, used to practice methods as provided herein, are formulated for administration by any or a variety of means including orally, parenterally, by inhalation spray, nasally, topically, intrathecally, intrathecally, intracerebrally, epidurally, intracranially or rectally. ADAR1 inhibiting agents, including drugs, vectors, liposomes, lipid nanoparticles (LNP), nanoliposomes or nanoparticles used to practice methods as provided herein, can further comprise pharmaceutically acceptable carriers, adjuvants and vehicles. In alternative embodiments, therapeutic combinations of drugs as provided herein, and drugs used to practice methods as provided herein, are formulated for parenteral administration, including administration intrathecally, intracerebrally or epidurally (into a intrathecal, intracerebral, epidural space), subcutaneously, intravenously, intramuscularly and/or intraarterially; for example, by injection routes but also including a variety of infusion techniques. Intraarterial, intrathecal, intracranial, epidural, intravenous and other injections as used in some embodiments can include administration through catheters or pumps, for example, an intrathecal pump, or an implantable medical device (which can be an intrathecal pump or catheter).

In alternative embodiments, ADAR1 inhibiting agents, including drugs, liposomes, lipid nanoparticles (LNP), nanoliposomes, vectors or nanoparticles, are formulated in accordance with a routine procedure(s) adapted for a desired administration route. In alternative embodiments, therapeutic combinations of drugs as provided herein, and drugs used to practice methods as provided herein, are formulated or manufactured as lyophilates, powders, lozenges, liposomes, lipid nanoparticles (LNP), nanoliposomes, suspensions, solutions or emulsions in oily or aqueous vehicles, and can contain formulatory agents such as suspending, stabilizing and/or dispersing agents.

In alternative embodiments, ADAR1 inhibiting agents, including drugs, liposomes, lipid nanoparticles (LNP), nanoliposomes, vectors or nanoparticles, can be formulated as a preparation for implantation or injection. Thus, for example, the compounds can be formulated with suitable polymeric or hydrophobic materials (for example, as an emulsion in an acceptable oil) or ion exchange resins, or as sparingly soluble derivatives (for example, as a sparingly soluble salt). Alternatively, the active ingredient can be in powder form for constitution with a suitable vehicle, for example, sterile pyrogen-free water, before use. Suitable alternative and exemplary formulations for each of these methods of administration can be found, for example, in Remington: The Science and Practice of Pharmacy, A. Gennaro, ed., 20th edition, Lippincott, Williams & Wilkins, Philadelphia, Pa.

In alternative embodiments, ADAR1 inhibiting agents, including drugs, liposomes, lipid nanoparticles (LNP), nanoliposomes, vectors or nanoparticles, are formulated with sterile water or saline, a polyalkylene glycol such as a polyethylene glycol, an oil of synthetic or vegetable origin, a hydrogenated naphthalene and the like. In alternative embodiments, therapeutic combinations of drugs as provided herein, and drugs used to practice methods as provided herein, can be formulated in or with a biocompatible, biodegradable lactide polymer, a lactide/glycolide copolymer, or polyoxyethylene-polyoxypropylene copolymers can be useful excipients to control the release of active compounds.

In alternative embodiments, ADAR1 inhibiting agents, including drugs, liposomes, lipid nanoparticles (LNP), nanoliposomes, vectors or nanoparticles, are administered using parenteral delivery systems such as ethylene-vinyl acetate copolymer particles, osmotic pumps, implantable infusion systems, intrathecal catheters, pumps and implants, and/or use of liposomes, lipid nanoparticles (LNP), nanoliposomes. Formulations for parenteral administration can also include glycocholate for buccal administration, methoxysalicylate for rectal administration, or citric acid for vaginal administration. Formulations for inhalation administration can contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally.

In alternative embodiments, ADAR1 inhibiting agents, including drugs, liposomes, lipid nanoparticles (LNP), nanoliposomes, vectors or nanoparticles, are administered intranasally. When given by this route, examples of appropriate dosage forms are a nasal spray or dry powder, as is known to those skilled in the art. For example, a nasal formulation can comprise a conventional surfactant, generally a non-ionic surfactant. When a surfactant is employed in a nasal formulation, the amount present will vary depending on the particular surfactant chosen, the particular mode of administration (for example drop or spray) and the effect desired.

In alternative embodiments, ADAR1 inhibiting agents, including drugs, liposomes, lipid nanoparticles (LNP), nanoliposomes, vectors or nanoparticles, are in the form of a sterile injectable preparation, such as a sterile injectable aqueous or oleaginous suspension. This suspension may be formulated according to the known art using those suitable dispersing or wetting agents and suspending agents. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In alternative embodiments, sterile fixed oils are conventionally be employed as a solvent or suspending medium. For this purpose any bland fixed oil may be employed including synthetic mono- or diglycerides. In alternative embodiments, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule (ampoule) or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

In alternative embodiments, ADAR1 inhibiting agents, including drugs, liposomes, lipid nanoparticles (LNP), nanoliposomes, vectors or nanoparticles, further comprise aqueous and non-aqueous sterile injection solutions that can contain (comprise) antioxidants, buffers, bacteriostats, bactericidal antibiotics and solutes that render the formulation isotonic with the bodily fluids of the intended recipient; and/or aqueous and non-aqueous sterile suspensions, which can include suspending agents and thickening agents.

In alternative embodiments, ADAR1 inhibiting agents, including drugs, liposomes, lipid nanoparticles (LNP), nanoliposomes, vectors or nanoparticles, are formulated for topical administration, for example, in the form of a liquid, lotion, cream or gel. Topical administration can be accomplished by application directly on the treatment area. For example, such application can be accomplished by rubbing the formulation (such as a lotion or gel) onto the skin of the treatment area, or by a spray application of a liquid formulation onto the application or treatment area.

In alternative embodiments, ADAR1 inhibiting agents, including drugs, liposomes, lipid nanoparticles (LNP), nanoliposomes, vectors or nanoparticles, comprise a bioimplant or a bioimplant material, and also can be coated with a compound of the invention or other compounds so as to improve interaction between cells and the implant.

In alternative embodiments, ADAR1 inhibiting agents, including drugs, liposomes, lipid nanoparticles (LNP), nanoliposomes, vectors or nanoparticles, comprise minor amounts of wetting or emulsifying agents, or pH buffering agents.

In alternative embodiments, ADAR1 inhibiting agents, including drugs, liposomes, lipid nanoparticles (LNP), nanoliposomes, vectors or nanoparticles, are formulated as a suppository, with traditional binders and carriers such as triglycerides.

In alternative embodiments, ADAR1 inhibiting agents, including drugs, liposomes, lipid nanoparticles (LNP), nanoliposomes, vectors or nanoparticles, comprise oral formulations such as tablets, pills, troches, lozenges (see, for example, as described in U.S. Pat. No. 5,780,055), aqueous or oil suspensions, dispersible powders or granules, emulsions, hard or soft capsules or geltabs, gels, jellies, syrups and/or elixirs. Compositions intended for oral use may be prepared according to any method known to the art for the manufacture of pharmaceutical compositions and such compositions may contain one or more agents including sweetening agents, taste-masking agents, flavoring agents, coloring agents and preserving agents, in order to provide a palatable preparation. Oral formulations can include standard carriers such as pharmaceutical grades of mannitol, lactose, starch, magnesium stearate, polyvinyl pyrrolidone, sodium saccharine, cellulose, magnesium carbonate, etc. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In alternative embodiments, formulations for oral use are hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

In alternative embodiments, ADAR1 inhibiting agents, including drugs, liposomes, lipid nanoparticles (LNP), nanoliposomes, vectors or nanoparticles, comprise aqueous suspensions comprising the active material in admixture with excipients suitable for the manufacture of aqueous suspensions. Exemplary excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (for example, lecithin), a condensation product of an alkylene oxide with a fatty acid (for example, polyoxyethylene stearate), a condensation product of ethylene oxide with a long chain aliphatic alcohol (for example, heptadecaethyleneoxycetanol), a condensation product of ethylene oxide with a partial ester derived from a fatty acid and a hexitol anhydride (for example, polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

In alternative embodiments, ADAR1 inhibiting agents, including drugs, liposomes, lipid nanoparticles (LNP), nanoliposomes, vectors or nanoparticles, comprise oil suspensions that can be formulated by suspending the active ingredient (for example, a compound of this invention) in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

In alternative embodiments, ADAR1 inhibiting agents, including drugs, liposomes, lipid nanoparticles (LNP), nanoliposomes, vectors or nanoparticles, include an agent which controls release of the compound, thereby providing a timed or sustained release compound.

In alternative embodiments, ADAR1 inhibiting agents, including drugs, liposomes, lipid nanoparticles (LNP), nanoliposomes, vectors or nanoparticles, are formulated or made as a multiparticulate and/or a solid dispersion formulation, for example, as described in, for example, U.S. Patent App. Pub. No. 20080118560, for example, comprising a hydrophobic matrix former which is a water-insoluble, non-swelling amphiphilic lipid; and a hydrophilic matrix former which is a meltable, water-soluble excipient. In one embodiment, ADAR1 inhibiting agents, including drugs, liposomes, lipid nanoparticles (LNP), nanoliposomes, vectors or nanoparticles, are contained in tablets, pills, capsules, troches, and the like comprising any combination of a binder, for example, as a starch, polyvinyl pyrrolidone, gum tragacanth or gelatin; a filler, such as microcrystalline cellulose or lactose; a disintegrating agent, such as crospovidone, sodium starch glycolate, corn starch, and the like; a lubricant, such as magnesium stearate, stearic acid, glyceryl behenate; a glidant, such as colloidal silicon dioxide and talc; a sweetening agent, such as sucrose or saccharin, aspartame, acesulfame-K; and/or flavoring agent, such as peppermint, methyl salicylate, or orange flavoring. When the dosage unit form is a capsule, it also can comprise a liquid carrier, such as a fatty oil.

In alternative embodiments, ADAR1 inhibiting agents, including drugs, liposomes, lipid nanoparticles (LNP), nanoliposomes, vectors or nanoparticles used to practice methods as provided herein, comprise (or are contained or packaged in) unit dosage formulations having a coating, for example, a coat comprising a sugar, shellac, sustained and/or other enteric coating agents, or any pharmaceutically pure and/or nontoxic agents.

In alternative embodiments, ADAR1 inhibiting agents, including drugs, liposomes, lipid nanoparticles (LNP), nanoliposomes, vectors or nanoparticles used to practice methods as provided herein, comprise (or are contained or packaged in) unit dosage formulations, wherein each different compound of the composition or product of manufacture is contained in a different layer of a pill, tablet or capsule, for example, as described in U.S. Pat. No. 7,384,653, for example, having an outer base-soluble layer and an inner acid-soluble layer. In alternative embodiments, therapeutic combinations of drugs as provided herein, and drugs used to practice methods as provided herein, comprise (or are contained or packaged in) unit dosage formulations, wherein each different compound of the composition or product of manufacture is contained in a liquid or a gel of different viscosity, for example, described in U.S. Patent App. Pub. No. 20050214223. In alternative embodiments, ADAR1 inhibiting agents, including drugs, liposomes, lipid nanoparticles (LNP), nanoliposomes, vectors or nanoparticles used to practice methods as provided herein, comprise (or are contained or packaged in) unit dosage formulations having reduced abuse potential, for example, as described in U.S. Patent App. Pub. No. 20040228802, for example, comprising a bittering agent, a bright deterrent/indicator dye, or a fine insoluble particulate matter.

### Carriers

In alternative embodiments, ADAR1 inhibiting agents, including drugs, liposomes, lipid nanoparticles (LNP), nanoliposomes, vectors or nanoparticles used to practice methods as provided herein, comprise or are formulated with or as aqueous or non-aqueous solutions, suspensions, emulsions and solids. Examples of non-aqueous solvents suitable for use as disclosed herein include, but are not limited to, propylene glycol, polyethylene glycol, vegetable oils such as olive oil, and injectable organic esters such as ethyl oleate. In alternative embodiments, aqueous carriers can comprise water, ethanol, alcoholic/aqueous solutions, glycerol, emulsions and/or suspensions, including saline and buffered media. Oral carriers can be elixirs, syrups, capsules, tablets and the like.

In alternative embodiments, liquid carriers are used to manufacture or formulate ADAR1 inhibiting agents, including drugs, liposomes, lipid nanoparticles (LNP), nanoliposomes, vectors or nanoparticles used to practice methods as provided herein, including carriers for preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can comprise other suitable pharmaceutical additives such as solubilizers, emulsifiers, buffers, preservatives, sweeteners, flavoring agents, suspending agents, thickening agents, colors, viscosity regulators, stabilizers or osmo-regulators.

In alternative embodiments, liquid carriers used to manufacture or formulate compounds of this invention comprise water (partially containing additives as above, for example cellulose derivatives, alternatively sodium carboxymethyl cellulose solution), alcohols (including monohydric alcohols and polyhydric alcohols, for example glycols) and their derivatives, and oils (for example fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

In alternative embodiments, solid carriers are used to manufacture or formulate ADAR1 inhibiting agents, including drugs, liposomes, lipid nanoparticles (LNP), nanoliposomes, vectors or nanoparticles used to practice methods as provided herein, including solid carriers comprising substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (for example, povidone, gelatin, hydroxypropylmethyl cellulose), lubricant, inert diluent, preservative, disintegrant (for example, sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

In alternative embodiments, parenteral carriers are used to manufacture or formulate ADAR1 inhibiting agents, including drugs, liposomes, lipid nanoparticles (LNP), nanoliposomes, vectors or nanoparticles used to practice methods as provided herein, including parenteral carriers suitable for use as disclosed herein include, but are not limited to, sodium chloride solution, Ringer's dextrose, dextrose and sodium chloride, lactated Ringer's and fixed oils. Intravenous carriers can comprise fluid and nutrient replenishers, electrolyte replenishers such as those based on Ringer's dextrose and the like. Preservatives and other additives can also comprise, for example, antimicrobials, antioxidants, chelating agents, inert gases and the like.

In alternative embodiments, carriers used to manufacture or formulate ADAR1 inhibiting agents, including drugs, liposomes, lipid nanoparticles (LNP), nanoliposomes, vectors or nanoparticles used to practice methods as provided herein, can be mixed as needed with disintegrants, diluents, granulating agents, lubricants, binders and the like using conventional techniques known in the art. The carriers can also be sterilized using methods that do not deleteriously react with the compounds, as is generally known in the art.

The invention also provides articles of manufacture and kits containing (comprising) ADAR1 inhibiting agents, including drugs, liposomes, lipid nanoparticles (LNP), nanoliposomes, vectors or nanoparticles used to practice methods as provided herein, including pharmaceutical compositions and formulations. By way of example only a kit or article of manufacture can include a container (such as a bottle) with a desired amount of a compound (or pharmaceutical composition of a compound) described herein. Such a kit or article of manufacture can further include instructions for using the ADAR1 inhibiting agents, including drugs, liposomes, lipid nanoparticles (LNP), nanoliposomes, vectors or nanoparticles, as described herein. The instructions can be attached to the container, or can be included in a package (such as a box or a plastic or foil bag) holding the container.

The ADAR1 inhibiting agents, including drugs, liposomes, lipid nanoparticles (LNP), nanoliposomes, vectors or nanoparticles used to practice methods as provided herein, can be delivered to the body or targeted to a specific tissue or organ (for example, a muscle or a brain) by any method or protocol, for example, including ex vivo “loading of cells” with therapeutic combinations of drugs as provided herein, and drugs used to practice methods as provided herein, where the “loaded cell” is the administered intramuscularly, or intrathecally, intracerebrally, or epidurally into the central nervous system (CNS), for example, as described in U.S. Pat. App. Pub. No. 20050048002.

In alternative embodiments, ADAR1 inhibiting agents, including drugs, liposomes, lipid nanoparticles (LNP), nanoliposomes, vectors or nanoparticles used to practice methods as provided herein, are first lyophilized and then suspended in a hydrophobic medium, for example, comprising aliphatic, cyclic or aromatic molecules, for example, as described in U.S. Pat. App. Pub. No. 20080159984.

In alternative embodiments, ADAR1 inhibiting agents, including drugs, liposomes, lipid nanoparticles (LNP), nanoliposomes, vectors or nanoparticles used to practice methods as provided herein, comprise or are formulated as pharmaceutically acceptable salts. Pharmaceutically acceptable salts can include suitable acid addition or base salts thereof. In alternative embodiments, compounds can be formulated as described in Berge et al, J Pharm Sci, 66, 1-19 (1977).

In alternative embodiments, ADAR1 inhibiting agents, including drugs, liposomes, lipid nanoparticles (LNP), nanoliposomes, vectors or nanoparticles used to practice methods as provided herein, are formulated as salts that are formed, for example, with strong inorganic acids such as mineral acids, for example hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkane-carboxylic acids of 1 to 4 carbon atoms which are unsubstituted or substituted (for example, by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with amino acids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Compounds of the invention also encompass salts which are not pharmaceutically acceptable, for example, a salt may still be valuable as an intermediate in a synthetic or analytical process or protocol.

In alternative embodiments, ADAR1 inhibiting agents, including drugs, liposomes, lipid nanoparticles (LNP), nanoliposomes, vectors or nanoparticles used to practice methods as provided herein, comprise any acceptable salt for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. Pharmaceutical compositions as disclosed herein can be prepared in accordance with methods well known and routinely practiced in the art. See, for example, Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978.

In some embodiments, ADAR1 inhibiting agents, including drugs, liposomes, lipid nanoparticles (LNP), nanoliposomes, vectors or nanoparticles as provided herein, are provided in the form of pharmaceutically acceptable salts comprising an amine that is basic in nature and can react with an inorganic or organic acid to form a pharmaceutically acceptable acid addition salt; for example, such salts comprise inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids; or optionally such pharmaceutically acceptable salts comprise sulfate, pyrosulfate, bisulfate, sulfite, bisulfite, phosphate, mono-hydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, beta-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like salts.

In alternative embodiments, ADAR1 inhibiting agents, including drugs, liposomes, lipid nanoparticles (LNP), nanoliposomes, vectors or nanoparticles used to practice methods as provided herein, comprise compositions manufactured under “Good manufacturing practice” or GMP, or “current good manufacturing practices” (cGMP), conditions.

### Methods of Administration

In alternative embodiments, ADAR1 inhibiting agents, including drugs, liposomes, lipid nanoparticles (LNP), nanoliposomes, vectors or nanoparticles used to practice methods as provided herein, are administered by any or a variety of means including orally, parenterally, by inhalation spray, nasally, topically, intrathecally, intrathecally, intracerebrally, epidurally, intracranially or rectally. ADAR1 inhibiting agents, including drugs, liposomes, lipid nanoparticles (LNP), nanoliposomes, vectors or nanoparticles used to practice methods as provided herein, can be administered with pharmaceutically acceptable carriers, adjuvants and vehicles. In alternative embodiments, ADAR1 inhibiting agents, including drugs, liposomes, lipid nanoparticles (LNP), nanoliposomes, vectors or nanoparticles used to practice methods as provided herein, are administered by injection routes, including a variety of infusion techniques. Intraarterial, intrathecal, intracranial, epidural, intravenous and other injections can include administration through catheters or pumps, for example, an intrathecal pump, or an implantable medical device (which can be an intrathecal pump or catheter).

In alternative embodiments, ADAR1 inhibiting agents, including drugs, liposomes, lipid nanoparticles (LNP), nanoliposomes, vectors or nanoparticles used to practice methods as provided herein, are administered by any known method or route, including by intranasal, intramuscular, intravenous, topical or oral, or combinations thereof, routes.

One embodiment comprises a product of manufacture comprising a pharmaceutical composition or a formulation, a blister package, a lidded blister or a blister card or packet, a clamshell, a tray or a shrink wrap, or a kit, comprising: ADAR1 inhibiting agent, including drug, vector or nanoparticle preparations as provided herein for oral administration.

In alternative embodiments, although all ingredients can be in one blister package, a lidded blister or a blister card or packet, a clamshell, a tray or a shrink wrap, or a kit, separate ingredients can be formulated for example, for topical application, for oral or for topical application. Each ingredient can be either separately packaged, or can be formulated as one unit dose, for example, as one tube (for example, with gel, lotion etc.), ampoule, blister packette and the like.

### Dosages

In alternative embodiments, ADAR1 inhibiting agents, including drugs, liposomes, lipid nanoparticles (LNP), nanoliposomes, vectors or nanoparticles used to practice methods as provided herein, are formulated and administered in a variety of different dosages and treatment regimens, depending on the disease or condition to be ameliorated, the condition of the individual to be treated, the goal of the treatment, and the like, as to be routinely determined by the clinician, see for example, the latest edition of Remington: The Science and Practice of Pharmacy, Mack Publishing Co., supra.

In alternative embodiments, an effective amount of ADAR1 inhibiting agents, including drugs, liposomes, lipid nanoparticles (LNP), nanoliposomes, vectors or nanoparticles used to practice the methods as provided herein, including a stereoisomer, salt, hydrate or solvate, is between about 0.1 mg and about 20.0 mg per kg of body weight of the individual or subject (for example, patient). In another variation, the effective amount is between about 0.1 mg and about 10.0 mg per kg of body weight of the individual or subject (for example, patient) or between about 0.1 mg and about 5.0 mg per kg of body weight of the patient. Alternately, the effective amount is between about 0.2 mg and about 2 mg per kg of body weight of the individual or subject (for example, patient).

In alternative embodiments, an effective amount of an ADAR1 inhibiting agents, including drugs, liposomes, lipid nanoparticles (LNP), nanoliposomes, vectors or nanoparticles used to practice methods as provided herein (for example, as a solid dosage, such as a pill, tablet or lozenge) is between about 0.1 mg and about 10.0 mg per kg of body weight of said individual, subject or patient; or is between about 0.1 mg and about 2.0 mg per kg of body weight; or is about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, or about 1.0 mg, per kg of body weight; or an effective amount of a drug or compound as provided herein, or a composition used to practice the methods as provided herein, is about 0.1 mg, about 0.15 mg, about 0.2 mg, about 0.25 mg or about 0.3 mg per kg of body weight.

In alternative embodiment, an effective amount (for example, as a solid dosage, such as a pill, tablet or lozenge) of an ADAR1 inhibiting agent, including drugs, liposomes, lipid nanoparticles (LNP), nanoliposomes, vectors or nanoparticles used to practice the methods as provided herein, is between about 0.25 mg and about 100 mg, between about 0.5 mg and about 200 mg, or between about 1 mg and about 400 mg; or is a solid dosage form comprising between about is between about 0.25 mg and about 100 mg, between about 0.5 mg and about 200 mg, or between about 1 mg and about 250 mg; or the solid dosage form comprises between about 5 mg and about 150; or the solid dosage form (for example, as a pill, tablet or lozenge) comprises between about 1 mg and about 75; or the solid dosage form comprises about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, or about 75 mg.

### Nanoparticles, Nanolipoparticles and Liposomes

Provided are nanoparticles, nanolipoparticles, vesicles and liposomal membranes comprising compounds and compositions used to practice the methods and embodiments as provided herein, including for example, an ADAR1 inhibiting agent. Provided are multilayered liposomes, lipid nanoparticles (LNP), nanoliposomes comprising compounds used to practice embodiments as provided herein, for example, as described in Park, et al., U.S. Pat. Pub. No. 20070082042. The multilayered liposomes, lipid nanoparticles (LNP), nanoliposomes can be prepared using a mixture of oil-phase components comprising squalane, sterols, ceramides, neutral lipids or oils, fatty acids and lecithins, to about 200 to 5000 nm in particle size, to entrap a composition used to practice embodiments as provided herein.

Liposomes, lipid nanoparticles (LNP), nanoliposomes can be made using any method, for example, as described in Park, et al., U.S. Pat. Pub. No. 20070042031, including the method of producing a liposome by encapsulating an active agent (for example, an ADAR1 inhibiting agent, or any compound used to practice methods as provided herein), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice embodiments as provided herein comprise a substituted ammonium and/or polyanions, for example, for targeting delivery of a compound as provided herein, or a compound used to practice methods as provided herein, to a desired cell type or organ, for example, brain, as described for example, in U.S. Pat. Pub. No. 20070110798.

Provided are nanoparticles comprising compounds as provided herein, for example, used to practice methods as provided herein in the form of active agent-containing nanoparticles (for example, a secondary nanoparticle), as described, for example, in U.S. Pat. Pub. No. 20070077286. In one embodiment, provided are nanoparticles comprising a fat-soluble active agent used to practice embodiments as provided herein, or a fat-solubilized water-soluble active agent to act with a bivalent or trivalent metal salt.

In one embodiment, solid lipid suspensions can be used to formulate and to deliver compositions used to practice embodiments as provided herein to mammalian cells in vivo, in vitro or ex vivo, as described, for example, in U.S. Pat. Pub. No. 20050136121.

In alternative embodiments, ADAR1-encoding nucleic acids, or vectors used to practice methods as provided herein, are delivered in vivo using methods as provided herein can be in the form of, or comprise, an RNA, for example, mRNA, which can be formulated in a lipid formulation or a liposome and injected for example intramuscularly (IM), for example using formulations and methods as described in U.S. patent application no. US20210046173 A1, which describes delivering to a subject (for example, via intramuscular administration) an ADAR1-encoding nucleic acid that comprises a RNA (for example, mRNA) that comprises an open reading frame (ORF) that comprises (or consists of, or consists essentially of) or encodes for an ADAR1-encoding nucleic acid; wherein optionally the RNA (or the DNA-carrying expression vehicle) is formulated in a liposome, or a lipid nanoparticle (LNP), or nanoliposome, that comprises: non-cationic lipids comprise a mixture of cholesterol and DSPC, or a PEG-lipid, or PEG-modified lipid, or LNP, or an ionizable cationic lipid; or a mixture of (13Z,16Z)-N,N-dimethyl-2-nonylhenicosa-12,15-dien-1-amine, cholesterol, DSPC, and PEG-2000 DMG. In alternative embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG). In alternative embodiments, the LNP comprises 20-99.8 mole % ionizable cationic lipids, 0.1-65 mole % non-cationic lipids, and 0.1-20 mole % PEG-lipid. In alternative embodiments, the LNP comprises an ionizable cationic lipid selected from the group consisting of (2S)-1-({6-[(3))-cholest-5-en-3-yloxy]hexyl}oxy)-N,N-dimethyl-3-[(9Z)-octadec-9-en-1-yloxy]propan-2-amine; (13Z,16Z)−N,N-dimethyl-3-nonyldocosa-13,16-dien-1-amine; and N,N-dimethyl-1-[(1S,2R)-2-octylcyclopropyl]heptadecan-8-amine; or a pharmaceutically acceptable salt thereof, or a stereoisomer of any of the foregoing. In alternative embodiments, the PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In alternative embodiments, the ionizable cationic lipid comprises: 2,2-dilinoleyl-4-dimethylaminoethyl-[1,3]-dioxolane (DLin-KC2-DMA), dilinoleyl-methyl-4-dimethylaminobutyrate (DLin-MC3-DMA), di ((Z)-non-2-en-1-yl) 9-((4-(dimethylamino) butanoyl)oxy) heptadecanedioate (L319), (13Z,16Z)-N,N-dimethyl-3-nonyldocosa-13,16-dien-1-amine, (12Z,15Z)-N,N-dimethyl-2-nonylhenicosa-12,15-dien-1-amine, and N,N-dimethyl-1-[(1S,2R)-2-octylcyclopropyl] heptadecan-8-amine. In one embodiment, the lipid is (13Z,16Z)-N,N-dimethyl-3-nonyldocosa-13,16-dien-1-amine or N,N-dimethyl-1-[(1S,2R)-2-octylcyclopropyl] heptadecan-8-amine, each of which are described in PCT/US2011/052328, the entire contents of which are hereby incorporated by reference. In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### Delivery Vehicles

In alternative embodiments, any delivery vehicle can be used to practice the methods as provided herein, for example, to deliver compounds and compositions as provided herein, or a compound used to practice methods as provided herein, for example, an ADAR1 inhibiting agent, to mammalian cells, for example, in vivo, in vitro or ex vivo. For example, delivery vehicles comprising polycations, cationic polymers and/or cationic peptides, such as polyethyleneimine derivatives, can be used for example as described, for example, in U.S. Pat. Pub. No. 20060083737.

In one embodiment, a dried polypeptide-surfactant complex is used to formulate compounds and compositions as provided herein, or a compound used to practice embodiments as provided herein, for example as described, for example, in U.S. Pat. Pub. No. 20040151766.

In one embodiment, an ADAR1 inhibiting agents used to practice methods as provided herein, can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, for example, using any electroporation system as described for example in U.S. Pat. Nos. 7,109,034; 6,261,815; 5,874,268.

### Products of Manufacture, Formulations and Kits

Provided are products of manufacture, formulations, pharmaceutical compositions or formulations, and kits for practicing methods as provided herein, including components and compositions for practicing methods as provided herein, for example, comprising an ADAR1 (adenosine deaminase associated with RNA1) inhibiting agent, such as for example, comprising: a JAK2 inhibitor such as fedratinib, or INREBIC™, or ruxolitinib, or JAKAFI™, or a STAT3 inhibitor, or 8-aza-adenosine, a nucleoside analog or an integrase inhibitor, or raltegravir (or ISENTRESS™) or dolutegravir (or TIVICAY™), or a lentiviral shRNA ADAR1 knockdown vector, or a lentiviral ADAR1 mutant vector, or a lentiviral ADAR1 Z alpha domain deleted vector, or an interferon inhibitory compound, or a lentiviral ADAR1 or lentiviral ADAR1 shRNA; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Example 1: Purification and Production of Human Functional Anti-Viral RNA Editing Enzymes, ADAR1 and Related Lentiviral Vectors, Editing Reporters and Compounds

This example demonstrates that methods as provided herein using the exemplary methods are effective and can be used for eradicating or reducing the in vivo numbers of cancer stem cells.

### Methods

### Human ADAR1 Catalytic Domain (CD) Purification Protocol

Yeast Strain: Saccharomyces cerevisiae BJ2168
(From Goode lab/Expression vector: pEG(KT)
(URA/LEU minus) (From Zakarian lab/Princeton)
Gene: Codon Optimized ADAR1-CD (GenScript)

### Yeast Growth Media (1 Liter)

Minimal Selection Media (URA/LEU Minus Growth Media): 100 mM Potassium
Phosphate pH 6.0, 6.7 g Yeast Nitrogen Base, 1.92 g Synthetic Amino Acid Drop-Out
Mix (Minus URA/LEU), 5.0 g Ammonium Sulfate, 10.0 g
Succinic Acid, 2% Glycerol, 3% Lactic Acid, 2% Raffinose. pH media to pH 6.0 using NaOH pellets and sterilize using 0.22 um filter.
5× Induction Media: 50 g/L Select Yeast Extract, 100 g/L Bacto-Tryptone, 10% D(+)-Galactose. Filter media using 0.22 um sterile filter.

### Purification Buffers

Yeast “Popcorn” Buffer: 20 mM Hepes pH 8.0, 150 mM NaCl
   Add fresh 1 mM PMSF and 1 Roche protease inhibitor cocktail pill
YeastBuster Lysis Buffer (Novagen)
   Add 1×THP, 1 mM 1,4-dithiothreitol (DTT), 1 mM phenylmethanesulfonyl fluoride (PMSF), 1 Roche protease cocktail pill;

### GST-Pulldown Buffers

GST Binding Buffer: 20 mM Hepes pH 8.0, 150 mM NaCl, 0.1% Triton X100, 5%
Glycerol, 1 mM DTT
   Add fresh 1 mM PMSF and 1 Roche protease inhibitor cocktail pill High-Salt Wash Buffer: 20 mM Hepes pH 8.0, 500 mM NaCl, 0.1% Triton X100, 5% Glycerol, 1 mM DTT Low-Salt Wash Buffer: 20 mM Hepes pH 8.0, 75 mM NaCl, 0.1% Triton X100, 5% Glycerol, 1 mM DTT

### HiTrap Heparin Column (GE) Buffers

Buffer A: 20 mM Hepes pH 8.0, 75 mM NaCl, 5%
Glycerol, 1 mM DTT, 0.22 μm filtered Buffer B: 20 mM
Hepes pH 8.0, 1M NaCl, 5% Glycerol, 1 mM DTT,
0.22 μm filtered
Dialysis Buffer/Protein Storage Buffer: 20 mM Hepes pH 8.0, 150 mM NaCl, 5% Glycerol, 1 mM DTT

### Superdex 200 10/300 GL (GE) Buffer

Buffer A: 20 mM Hepes pH 8.0, 150 mM NaCl, 5% Glycerol, 1 mM DTT, 0.22 um filtered

### Yeast Growth Procedure

1.) In a sterile 150 mL baffled flask, inoculate 15 mL of Minimal Selection Media (URAILEU minus media) with a large, single yeast colony picked from a freshly streaked URAILEU minus plate. Grow culture overnight @ 30° C. in shaker set to 250 RPM. Note: This protocol is designed for the preparation of 3 liters of yeast culture.
2.) The next day, inoculate 800 mL of Minimal Selection Media in 2-liter baffled flasks with 1.5 mL of starter culture each. Grow cultures overnight @ 30° C. in shaker set to 250 RPM.
3.) The next morning, use a Spectronic 200 spectrophotometer to measure the optical density (OD) of the cultures using a wavelength of 600 nm. For ideal gene expression, the OD600 nm should be between 1.0 and 2.0. To induce protein expression, add 200 mL of 5× Induction Media to each flask. Allow yeast to grow
   @ 30 degrees C. for 24 hours in shaker set to 250 RPM.
4.) Harvest yeast by spinning cultures down @ 5K RPM for 10 minutes using 500 mL spin bottles. Repeat this step until all of the culture is pelleted.
5.) Discard the supernatant and combine pellets by resuspending them in Yeast “Popcorn” Buffer. First, resuspend pellets by vortexing them in 20 mL of buffer. Next, transfer yeast into a 50 ml conical tube, and re-pellet the yeast by using a table-top centrifuge @ 5K RPM for 10 minutes. Discard wash and save pellet.
6.) Resuspend pellet in Yeast “Popcorn” Buffer containing 1 mM PMSF and 1 Roche protease inhibitor cocktail pill. For resuspension, use half the pellet volume and fully resuspend the yeast by vortexing (for example Use 5 mL of popcorn buffer for a 10 mL wet yeast pellet). Note: Roche protease inhibitor cocktail pills are pre-solubilized in popcorn buffer before use hereon in through this protocol.
7.) Lastly, make yeast “popcorn” by adding the yeast to liquid nitrogen drop-by-drop in a 50 ml conical tube.
   Store popcorn @−80 degrees C. for long-term storage.

### Yeast Lysis Protocol

1.) Begin lysis protocol by pre-chilling ceramic mortar and pestle. To do so, place mortar in a bucket containing dry ice. Make sure to completely cover the sides of the mortar with dry ice. Additionally, fill the mortar with liquid nitrogen, and allow the liquid nitrogen to dissipate completely before use. The pestle and spatula should also be pre-chilled by submerging them in liquid nitrogen prior to Step 2. Note: Always use blue cryogenic gloves when handling liquid nitrogen.
2.) Next, place a small amount of yeast popcorn in the mortar and begin grinding by carefully maneuvering the pestle in a circular motion until a very fine powder is formed. This may take up to 10 minutes per round of grinding.
3.) Transfer ground yeast material, or “powder”, to a new 50 mL conical tube. Yeast powder can be stored at −80 degrees C. before commencing the next step if needed.
4.) Thaw out yeast powder at room temperature. While the powder is thawing, activate 100 mL of YeastBuster Lysis Buffer. To activate the buffer, add 1 mL of 100×THP, 1 mM DTT, 1 mM PMSF, 100 uL cyanase (50 U/uL), and 1 Roche protease inhibitor cocktail pill.
5.) Resuspend the powder in all 100 mL activated Yeast Buster Lysis Buffer. DO NOT SHAKE OR VORTEX SAMPLE AS THIS WILL DAMAGE YOUR PROTEIN AND CAUSE FOAM TO FORM!
6.) Place sample to a 500 ml beaker with a stir bar and allow it stir at 100 RPM @ room temperature for 1 hour.
7.) Complete the lysis protocol by douncing the sample 30 times in a pre-chilled dounce homogenizer. This can be done in a piece-wise fashion douncing 15 mL of sample at a time.
8.) Lastly, pellet the unbroken cells and cell debris by spinning extract @ 15K RPM for 25 minutes in a chilled centrifuge. Note: Make sure to use tubes that will withstand these spin speeds (for example Nalgene centrifuge tubes).
9.) Discard the pellet and transfer the supernatant containing your protein to new 50 mL conical tubes. Measure the protein concentration of the extract and determine the total amount of protein in sample. Flash freeze extract using liquid nitrogen for later use. Store frozen extracts at −80° C.

### GST-ADAR1-CD Purification Protocol

1.) Begin by thawing protein extracts in room temperature preferably in water. Once thawed, dilute the extract in GST Binding Buffer to a total volume of 500 mL. Add fresh 1 mM DTT, 1 mM PMSF, and 1 Roche protease inhibitor cocktail pill to final diluted extract solution (Starting Material). Ideally, the final protein concentration of the extract will be between 1-2 mg/mL.
2.) While extracts are thawing out, pre-equilibrate GST resin with 150 mL (3 CV) of ice-cold GST Binding Buffer.
3.) Next, pass extract over the GST resin. Keep extract and flow through on ice throughout the entire process as this step takes several hours to complete. Additionally, save 100 μL of starting material and flow through for anti-ADAR1 or anti-GST Western blot analysis.
4.) After passing through the extract, beginning washing the GST resin with the following wash buffers:
a.) 100 mL GST Binding Buffer; b.) 100 mL High Salt Wash Buffer; c.) 100 mL GST Binding Buffer; d.) 100 mL Low Salt Buffer.
5.) After the last wash, remove the stop-cock and replace it with a small yellow cap and seal it well with parafilm.
6.) Add 50 mL of Low Salt Buffer, carefully resuspend the GST resin, and transfer all of the beads evenly into 4×50 mL conical tubes (˜25 mL/each tube).
7.) Fill each tube up to 50 mL with Low Salt Buffer containing 1 mM DTT. The final elution volume is 150 mL (approximately 3 CV).
8.) Add 300 uL TEV protease to each tube and incubate overnight at 4° C. rotating gently.
9.) The next day, transfer all of the GST resin and solution back into the purification column. Release/open the top screw cap first to release the internal pressure of the column. Then, carefully open the bottom screw cap and catch the TEV-eluted protein in a 250 ml beaker on ice. Save 160 μL of elution for protein concentration measurement and Coomassie Blue stain analysis.
10.) Proceed with purification by pumping the elution over 5 mL HiTrap Heparin columns (GE) attached in tandem to an AKTA Pure system (FPLC). Use the method script written on the AKTA Pure system. Note: The milligram quantity obtained from Step 9 will determine how many Heparin columns will be needed to capture all ADAR1-CD protein in the sample.
11.) Determine the separation of ADAR1-CD and other contaminating proteins including TEV protease by visualizing Heparin fractions on a SDS-PAGE gel and staining it with Coomassie Blue stain. Note: Determining the separation of proteins is critical before moving on to the next step.
12.) Collect FPLC fractions that contain ADAR1-CD, but not other contaminating proteins, and transfer them to a 50 ml conical tube on ice.
13.) Since the sample is in high salt (˜300 mM), the sample needs to be dialyzed in Dialysis Buffer using dialysis tubing that is pre-equilibrated in 1 mM EDTA pH 8.0 in dH20. Make sure that the dialysis tubing has a MWCO no larger than 10-14 kDa. Dialyze overnight at 4° C. in 3 liters of buffer stirring gently with a stir bar.
14.) The next morning, carefully transfer the sample to a new 50 mL conical tube on ice. At this point, the protein is ready to concentrate using a Millipore Amicon-Ultra centrifuge spin column with a MWCO of 10-14 kDa. Spin sample at 5,000×g for 20 minutes. After 20 minutes, take the sample out of the centrifuge and carefully pipette the protein solution to prevent undesired protein aggregation. Continue to spin sample until a final volume of 500 uL is achieved.
15.) Measure the protein concentration of the concentrated sample, save 20 uL for further SDS-PAGE analysis, and flash freeze the rest of the sample in liquid nitrogen. The sample is now ready for downstream applications. Note: Alternatively, the sample can be applied to a Superdex200 10/300 GL™ gel filtration column after the concentration step to further purify the protein.
Nano-Luciferase Reporter Design
In Vivo RNA Editase Reporter Construction

The RNA editase responsive reporter reporter was generated by sub cloning the following DNA sequence termed NanoLuc:

(SEQ ID NO: 2)
tctagaCTAGCCAAGGTGAGCGCGTCAATAAACATGCACGTTTATTAG
CGCGCCCACCCTGGAGCTAGCGTCTTCACACTCGAAGATTTCGTTGGG
GACTGGCGACAGACAGCCGGCTACAACCTGGACCAAGTCCTTGAACAG
GGAGGTGTGTCCAGTTTGTTTCAGAATCTCGGGGTGTCCGTAACTCCG
ATCCAAAGGATTGTCCTGAGCGGTGAAAATGGGCTGAAGATCGACATC
CATGTCATCATCCCGTATGAAGGTCTGAGCGGCGACCAAATGGGCCAG
ATCGAAAAAATTTTTAAGGTGGTGTACCCTGTGGATGATCATCACTTT
AAGGTGATCCTGCACTATGGCACACTGGTAATCGACGGGGTTACGCCG
AACATGATCGACTATTTCGGACGGCCGTATGAAGGCATCGCCGTGTTC
GACGGCAAAAAGATCACTGTAACAGGGACCCTGTGGAACGGCAACAAA
ATTATCGACGAGCGCCTGATCAACCCCGACGGCTCCCTGCTGTTCCGA
GTAACCATCAACGGAGTGACCGGCTGGCGGCTGTGCGAACGCATTCTG
gcggccgct

into pCDH-EF1-T2A-copGFP lentiviral expression plasmid (CD521A-1, SBI Systems Biosciences). Amplification of the NanoLuc sequence was generated using forward primer: GP17015XbaI/NanoLuc

(SEQ ID NO: 3)
(5′-ctagtctagactagccaaggtgagcgcgtca-3′)

and reverse primer GP17023NotI/NanoLuc

(SEQ ID NO: 4)
5′-atagtttagcggccgccagaatgcgttc gcacag-3′.

After amplification the pCDH-EF1-T2A-copGFP vector was digested with restriction enzymes XbaI and NotI. Ligation of the sequence above into XbaI/NotI digested pCDH-EF1-T2A-copGFP in-frame generated NanoLuc reporter responsive to RNA editase activity. A [TAG] stop codon is upstream of Nano luciferase and in response to the RNA editing of the adenine to inosine the codon is translated as [TGG] alleviating the stop codon block and inducing expression of the reporter NanoLuciferase. The housekeeping elongation factor 1α (EF1) promoter drives the expression of the reporter. Oligonucleotide primers were synthesized by Eton Bioscience (San Diego, CA). Verification of the NanoLuc reporter was completed using both restriction enzyme analysis and DNA sequencing.

### In Vivo Visualization of ADAR1 Editase Activity Via Nano-Luc Reporter

### Transduction

K562 cells from ATCC were initially transduced with control, ADAR1 WT, or ADAR1 E912A mutant vectors and maintained stably. These stable lines were then co-transduced with equal MOI of ADAR1 NanoLuc reporter lentivirus. Cells were then sub-cultured and maintained stably before transplantation into mice.

### Transplantation and Imaging

Immunocompromised RAG2−/−yc−/− mice were bred and housed in the Sanford Consortium vivarium per IACUC-approved protocol. Neonates (P2-P3) were transplanted intrahepatically with 100,000 K562 cells transduced with either pCDH, ADAR1 WT, or ADAR1 E912A vectors and ADAR1 NanoLuc reporter (all). Mice were monitored and weighed weekly after P21. Mice with >20% weight reduction (approximately 7 weeks old) compared to non-transplant control were imaged by IVIS lumina imaging system. Promega NANOLUC™ substrate was prepared at 40× (sterile PBS) and administered intraperitoneal at a volume (ul) equivalent to 10 times mouse weight (g). Mice were euthanized after imaging.

### FIGURE LEGENDS

FIG. 1. Expression and Purification of recombinant human ADAR1 Catalytic Domain (hADAR1 CD) in BJ2168 yeast expression system. (A) hADAR1 CD codon optimization for expression in yeast. (B) hADAR1 CD amino acid sequence. Colored amino acids have been deleted in Δloop construct. (C) pEG(KT) GST-TEV-hADAR1 CD and pEG(KT) GST-TEV-hADAR1 CD Δloop vector maps. (D) Schematic representation of Galactose-inducible expression system. (E) Coomassie Blue stain and α-ADAR1 Western Blot confirming Galactose-inducible expression of GST-tagged hADAR1 CD. (F) Workflow showing steps involved in protein purification from yeast cell extract. (G) Coomassie Blue stain showing successful cleavage of GST tag by TEV enzyme. (H) Silver stain demonstrating purity of the hADAR1 CD protein product after final purification step. (I) Size Exclusion Chromatography of purified hADAR1 CD using a Superdex200 10/300 GL gel filtration column.

(J) Protein mass determination of purified hADAR1 CD protein product via mass spectrometry. (K) Analytical Ultracentrifugation of purified hADAR1 CD demonstrating purity of the final protein product.

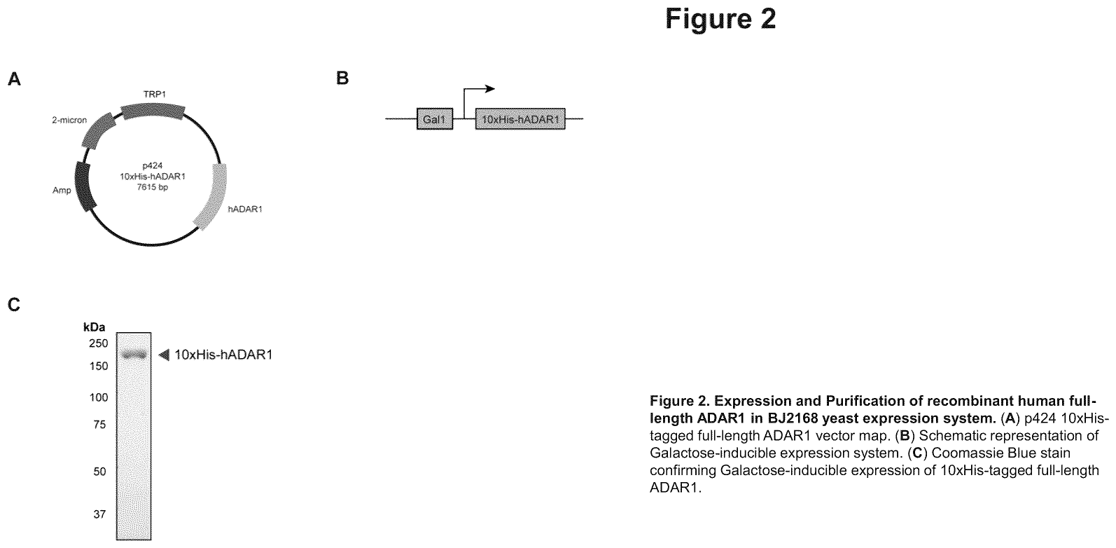

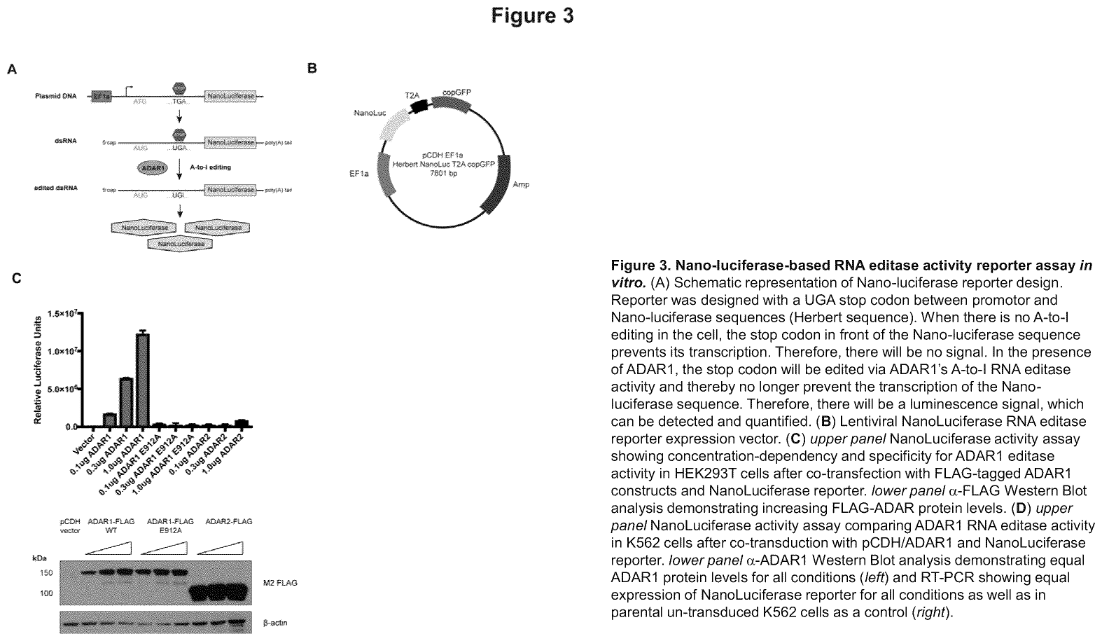

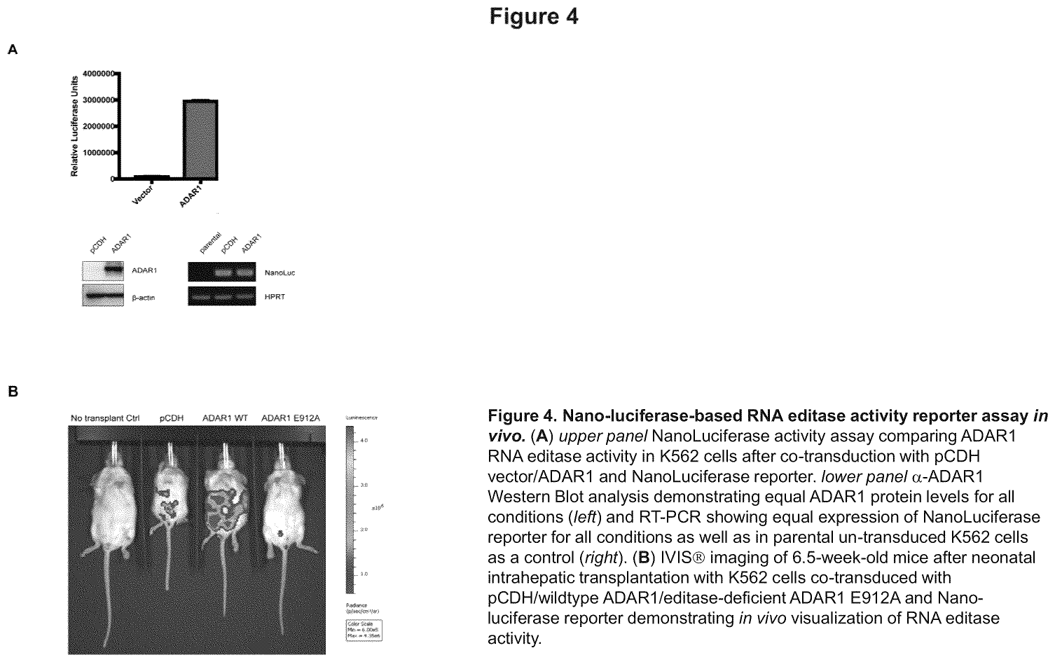

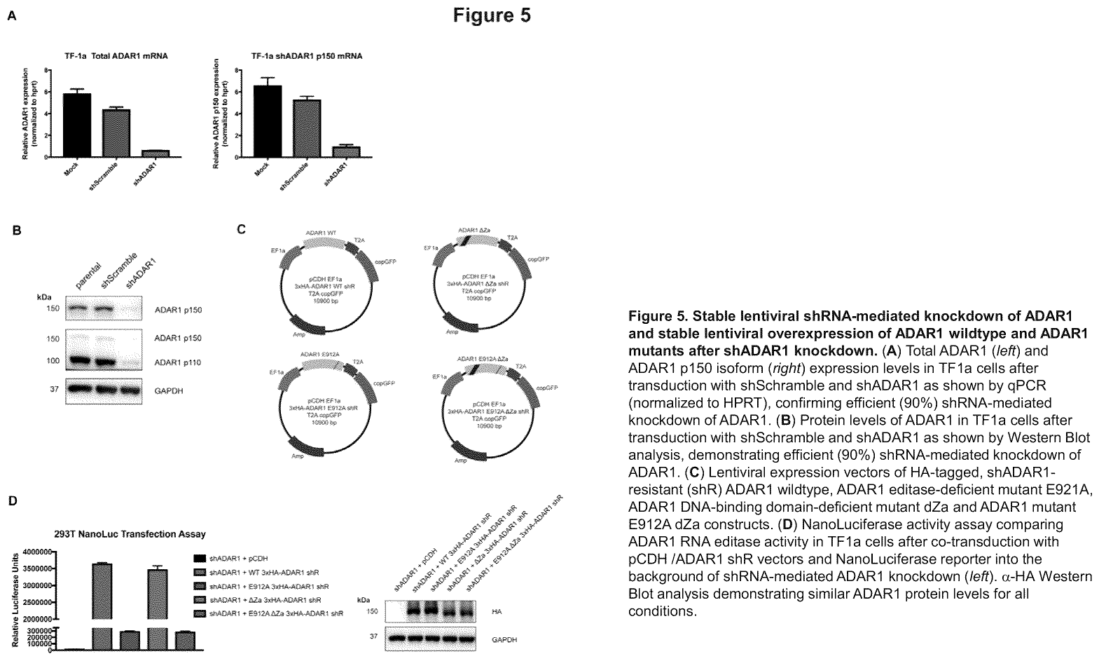

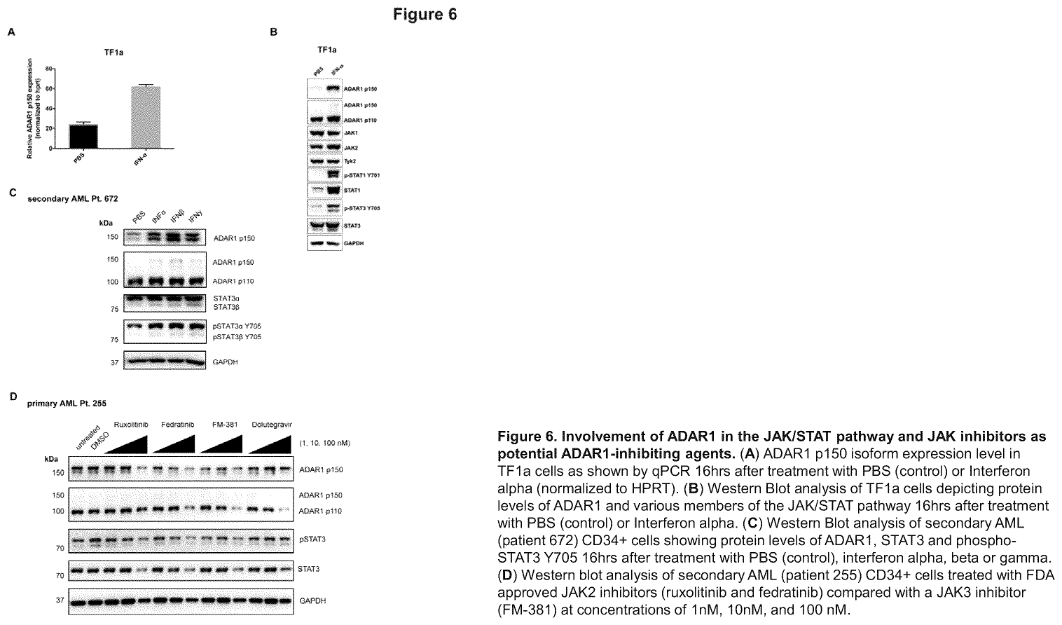

FIG. 2. Expression and Purification of recombinant human full-length ADAR1 in BJ2168 yeast expression system. (A) p424 10×His-tagged full-length ADAR1 vector map. (B) Schematic representation of Galactose-inducible expression system. (C) Coomassie Blue stain confirming Galactose-inducible expression of 10×His-tagged full-length ADAR1.

FIG. 3. Nano-luciferase-based RNA editase activity reporter assay in vitro. (A) Schematic representation of Nano-luciferase reporter design. Reporter was designed with a UGA stop codon between promotor and Nano-luciferase sequences (Herbert sequence). When there is no A-to-I editing in the cell, the stop codon in front of the Nano-luciferase sequence prevents its transcription. Therefore, there will be no signal. In the presence of ADAR1, the stop codon will be edited via ADAR1's A-to-I RNA editase activity and thereby no longer prevent the transcription of the Nano-luciferase sequence. Therefore, there will be a luminescence signal, which can be detected and quantified. (B) Lentiviral NanoLuciferase RNA editase reporter expression vector. (C) upper panel NanoLuciferase activity assay showing concentration-dependency and specificity for ADAR1 editase activity in HEK293T cells after co-transfection with FLAG-tagged ADAR1 constructs and NanoLuciferase reporter, lower panel a-FLAG Western Blot analysis demonstrating increasing FLAG-ADAR protein levels. (D) upper panel NanoLuciferase activity assay comparing ADAR1 RNA editase activity in K562 cells after co-transduction with pCDH/ADAR1 and NanoLuciferase reporter, lower panel α-ADAR1 Western Blot analysis demonstrating equal ADAR1 protein levels for all conditions (left) and RT-PCR showing equal expression of NanoLuciferase reporter for all conditions as well as in parental un-transduced K562 cells as a control (right).

FIG. 4. Nano-luciferase-based RNA editase activity reporter assay in vivo. (A) upper panel NanoLuciferase activity assay comparing ADAR1 RNA editase activity in K562 cells after co-transduction with pCDH vector/ADAR1 and NanoLuciferase reporter, lower panel α-ADAR1 Western Blot analysis demonstrating equal ADAR1 protein levels for all conditions (left) and RT-PCR showing equal expression of NanoLuciferase reporter for all conditions as well as in parental un-transduced K562 cells as a control (right). (B) IVIS™ imaging of 6.5-week-old mice after neonatal intrahepatic transplantation with K562 cells co-transduced with pCDH/wildtype ADAR1/editase-deficient ADAR1 E912A and Nano-luciferase reporter demonstrating in vivo visualization of RNA editase activity.

FIG. 5. Stable lentiviral shRNA-mediated knockdown of ADAR1 and stable lentiviral overexpression of ADAR1 wildtype and ADAR1 mutants after shADAR1 knockdown. (A) Total ADAR1 (left) and ADAR1 p150 isoform (right) expression levels in TF1a cells after transduction with shSchramble and shADAR1 as shown by qPCR (normalized to HPRT), confirming efficient (90%) shRNA-mediated knockdown of ADAR1. (B) Protein levels of ADAR1 in TF1a cells after transduction with shSchramble and shADAR1 as shown by Western Blot analysis, demonstrating efficient (90%) shRNA-mediated knockdown of ADAR1. (C) Lentiviral expression vectors of HA-tagged, shADAR1-resistant (shR) ADAR1 wildtype, ADAR1 editase-deficient mutant E921A, ADAR1 DNA-binding domain-deficient mutant dZa and ADAR1 mutant E912A dZa constructs. (D) NanoLuciferase activity assay comparing ADAR1 RNA editase activity in TF1a cells after co-transduction with pCDH/ADAR1 shR vectors and NanoLuciferase reporter into the background of shRNA-mediated ADAR1 knockdown (left), α-HA Western Blot analysis demonstrating similar ADAR1 protein levels for all conditions.

FIG. 6. Involvement of ADAR1 in the JAK/STAT pathway and JAK inhibitors as potential ADAR1-inhibiting agents. (A) ADAR1 p150 isoform expression level in TF1a cells as shown by qPCR 16 hrs after treatment with PBS (control) or Interferon alpha (normalized to HPRT). (B) Western Blot analysis of TF1a cells depicting protein levels of ADAR1 and various members of the JAK/STAT pathway 16 hrs after treatment with PBS (control) or Interferon alpha. (C) Western Blot analysis of secondary AML (patient 672) CD34+ cells showing protein levels of ADAR1, STAT3 and phospho-STAT3 Y705 16 hrs after treatment with PBS (control), interferon alpha, beta or gamma. (D) Western blot analysis of secondary AML (patient 255) CD34+ cells treated with FDA approved JAK2 inhibitors (ruxolitinib and fedratinib) compared with a JAK3 inhibitor (FM-381) at concentrations of 1 nM, 10 nM, and 100 nM.

A number of embodiments of the invention have been described. Nevertheless, it can be understood that various modifications may be made without departing from the spirit and scope of the invention. Accordingly, other embodiments are within the scope of the following claims.

1: A method for inhibiting an RNA virus or a retrovirus in vivo,
wherein optionally the RNA virus is a SARs-COV-2 virus,
the method comprising inhibiting an RNA virus or a retrovirus in an individual in need thereof in vivo, comprising administering a lentivirus expressing ADAR1 in vivo, resulting in lentiviral ADAR1 expression or overexpression in vivo,
wherein optionally the administering of the lentivirus expressing ADAR1 in vivo comprises intravenous (IV) administration of a lentiviral ADAR1 transduced stem cell, and optionally the stem cell is a cord blood CD34+ cell or a mesenchymal stromal cell.

2: A method for inhibiting replication of an RNA virus or a retrovirus,
wherein optionally the RNA virus is a SARs-COV-2 virus,
the method comprising inhibiting an RNA virus or a retrovirus in an individual in need thereof in vivo, comprising in vivo delivering or administering to the individual in need thereof an ADAR1 catalytic domain nanoprotein,
and optionally delivering or administering the ADAR1 catalytic domain nanoprotein contained in or formulated in a liposome, lipid nanoparticle (LNP), or nanoliposome, optionally delivering the ADAR1 catalytic domain nanoprotein by intravenous administration or by inhalation.

3-4. (canceled)

5: A method for eradicating or reducing in vivo numbers of cancer stem cells comprising administering to an individual in need thereof an ADAR1 (adenosine deaminase associated with RNA1) inhibiting agent, wherein the ADAR1 inhibiting agent reduces, or significantly reduces, ADAR1 levels in vivo,
wherein optionally the ADAR1 inhibiting agent reduces, or significantly reduces, ADAR1 Nano-luc reporter activity,
wherein optionally the ADAR1 inhibiting agent reduces, or significantly reduces, ADAR1 in cell lines and/or in human cancer stem cell assays.

6: The method of claim 5, wherein said ADAR1 inhibiting agent comprises a JAK2 inhibitor.

7: The method of claim 6, wherein the JAK2 inhibitor comprises fedratinib, or INREBIC™, or ruxolitinib, or JAKAFI™.

8: The method of claim 5, wherein said ADAR1 inhibiting agent comprises a STAT3 inhibitor.

9: The method of claim 5, wherein the ADAR1 inhibiting agent comprises 8-aza-adenosine, a nucleoside analog or an integrase inhibitor.

10: The method of claim 5, wherein the ADAR1 inhibiting agent comprises raltegravir (or ISENTRESS™) or dolutegravir (or TIVICAY™).

11: The method of claim 5, wherein the ADAR1 inhibiting agent comprises a lentiviral shRNA ADAR1 knockdown vector.

12: The method of claim 5, wherein the ADAR1 inhibiting agent comprises a lentiviral ADAR1 mutant vector.

13: The method of claim 5, wherein the ADAR1 inhibiting agent comprises a lentiviral ADAR1 Z alpha domain deleted vector.

14: The method of claim 5, wherein ADAR1 inhibiting agent comprises an interferon inhibitory compound.

15: The method of claim 5, wherein the ADAR1 inhibiting agent comprises lentiviral ADAR1 or lentiviral ADAR1 shRNA.

16: The method of claim 5, wherein the ADAR1 inhibiting agent comprises a recombinant human full length ADAR1.

17: The method of claim 6, wherein the ADAR1 inhibiting agent comprises a recombinant human ADAR1 catalytic domain.

18: The method of claim 5, wherein the ADAR1 inhibiting agent comprises a recombinant human Z alpha domain deleted ADAR1.

19: The method of claim 5, wherein the ADAR1 inhibiting agent comprises a JAK2-expressing vector, optionally a retroviral or a lentiviral JAK2 expression vector, optionally a retroviral or a lentiviral JAK2 overexpression vector.

20: The method of claim 5, wherein the ADAR1 inhibiting agent is formulated or manufactured as a parenteral formulation, an aqueous solution, a liposome, an injectable solution, a tablet, a pill, a lozenge, a capsule, a caplet, a spray, a sachet, an inhalant, a powder, a freeze-dried powder, an inhalant, a patch, a gel, a geltab, a nanosuspension, a nanoparticle, a nanoliposome, a microgel, a pellet, a suppository or any combination thereof,
and optionally the drug delivery device or product of manufacture is or comprises an implant.

21: The method of claim 5, wherein the ADAR1 inhibiting agent is are formulated or manufactured together in one parenteral formulation, one aqueous solution, one liposome, one injectable solution, one freeze-dried powder, one feed, one food, one food supplement, one pellet, one lozenge, one liquid, one elixir, one aerosol, one inhalant, one adhesive, one spray, one powder, one freeze-dried powder, one patch, one tablet, one pill, one capsule, one gel, one geltab, one lozenge, one caplet, one nanosuspension, one nanoparticle, one nanoliposome, one microgel or one suppository.

22: The method of claim 5, wherein the ADAR1 inhibiting agent is formulated in a unit dosage amount ranging from between 0.1 mg to about 1 gram, and optionally formulated as an immediate release formulation or a controlled release formulation.

23-31. (canceled)
